(19) United States
(12) Patent Application Publication
(10) Pub. No.: US 2024/0124832 A1
(43) Pub. Date: Apr. 18, 2024

(54) PRODUCTION OF TRICHODERMA SPP FUNGI USING ALMOND HULL AS A GROWTH MEDIUM

(71) Applicant: SELECT HARVESTS LIMITED, Thomastown (AU)

(72) Inventor: Upul GUNAWARDENA, Thomastown (AU)

(21) Appl. No.: 18/489,420

(22) Filed: Oct. 18, 2023

(30) Foreign Application Priority Data
Oct. 18, 2022 (AU) 2022903056

(51) Int. Cl. C12N 1/14

(52) U.S. Cl. CPC C12N 1/14

(57) ABSTRACT

The present invention relates to a method for producing Trichoderma spp. fungi comprising (i) combining shredded almond hull, a compost composition and Trichoderma spp. spores to form a mixture, (ii) incubating the mixture for a period of time sufficient to produce Trichoderma spp. fungi, and (iii) collecting the Trichoderma spp. fungi.

### CROSS-REFERENCE TO RELATED APPLICATIONS

The present application claims priority to Australian Provisional Patent Application No. 2022903056, filed 18 Oct. 2022, the entire disclosure of which is incorporated herein by cross-reference.

### FIELD OF THE DISCLOSURE

The present disclosure broadly relates to a method for producing a fungi using a growth medium comprising almond hull.

### BACKGROUND OF THE DISCLOSURE

Any discussion of the prior art throughout this specification should in no way be considered as an admission that such prior art is widely known or forms part of the common general knowledge in the field.

Trichoderma spp. are free living fungi that are present in healthy soil and root ecosystems. They are popular in both agriculture and horticulture because of their range of beneficial effects in soil.

Trichoderma spp. has been found to significantly suppress the growth of plant pathogenic microorganisms and to regulate the rate of plant growth. Common plant diseases such as root rot disease, damping off, wilt and fruit rot can be successfully controlled by Trichoderma spp.

Another important function of Trichoderma spp. is its symbiotic association with the root system of plants. The host plant provides soluble carbon to the fungi which is used for growth and metabolism, in exchange for the supply of nutrients. The fungi grow extensively through the soil and are able to reach well outside the root zone. They are also able to absorb more nutrients (such as phosphorous and trace elements such as zinc, manganese and iron) thereby facilitating an extension of the rootzone. This symbiotic association enhances root growth and is frequently associated with better crop productivity.

The fruit of the almond is a drupe comprising an outer hull and a hard shell. Inside the shell is the edible seed, which is commonly referred to as a nut, being the only edible part of the fruit. Generally, the hull comprises 50-60% (w/w) of the fruit, the shell 10-15% (w/w) and the seed less than 30% (w/w). Almond hull is a by-product resulting from isolation of the edible nut portion. Annually, the Australian almond industry produces over half a million metric tons of almond hull and shell. The majority of the almond hull is used as animal feed. However, the market for animal feed is inconsistent because it is dependent on rainfall. This creates an opportunity to use excess almond hull in alternative areas as a value-added product.

Against this background, the present inventors have developed a method for producing Trichoderma spp. fungi in which almond hull is used as part of a growth medium.

### SUMMARY OF THE DISCLOSURE

In a first aspect of the disclosure, there is provided a method for producing Trichoderma spp. fungi comprising
(i) combining shredded almond hull, a compost composition and Trichoderma spp. spores to form a mixture; and
(ii) incubating the mixture for a period of time sufficient to produce Trichoderma spp. fungi; and
(iii) collecting the Trichoderma spp. fungi.

The Trichoderma spp. fungi may be T. harzianum, T. viride, T. koningii or T. hamatum, or any other fungi of the Trichoderma genus.

The Trichoderma spp. fungi produced may comprise conidia and mycelia.

The ratio of the shredded almond hull to the compost composition to the Trichoderma spp. spores may be about [0.5 to 1.5]:[0.05 to 0.15]:[0.000115 to 0.000375] by weight, or about 1:0.1:0.00025 by weight.

The shredded almond hull may have a size between about 1 mm and about 10 mm, or between about 2 mm and about 5 mm.

The compost composition may be a composition produced by composting a blend comprising straw, horse or poultry manure and gypsum.

The compost composition may comprise one or more of the following:
a moisture content between about 50% (w/w) and 70% (w/w)
a pH between about 8.5 and about 10.5
an electrical conductivity between about 5 dS/m and about 20 dS/m.
a total carbon content between about 25% (w/w) and about 35% (w/w)
a total nitrogen content between about 1% (w/w) and about 5% (w/w)
a carbon:nitrogen ratio between about 5 and 15 (by weight)
organic matter in an amount between about 45% (w/w) and about 55% (w/w)
a nitrogen:sulfur ratio between about 1 and 2 (by weight)
a nitrogen:phosphorus ratio between about 2 and 4 (by weight)
a nitrogen:potassium ratio between about 0.5 and 2 (by weight)

The method may further comprise watering the mixture.

Watering the mixture may be performed one or more times during step (ii).

Watering the mixture may be performed prior to commencing step (ii).

The mixture may have a pH between about 6.6 and about 6.8.

The mixture may have a temperature between about 25° C. and about 27° C.

The mixture may have a moisture content between about 40% and about 80% (w/w), or between about 55% and about 65% (w/w).

The period of time may be between about 20 days and about 35 days, or between about 25 days and about 28 days.

In one embodiment step (i) comprises:
(a) forming a layered structure comprising a first layer which is the shredded almond hull, a second layer which is the compost composition and a third layer which is the Trichoderma spp. spores, and agitating the layered structure to form the mixture.

The shredded almond hull may form a bottom layer, the compost composition may form a middle layer and the Trichoderma spp. spores may form a top layer.

The mixture may be present as an elongate pile.

In one embodiment the method further comprises forming a depression over a length of the elongate pile.

The depression may be formed along a top section of the elongate pile.

The depression may be formed about 5 to 10 days after completion of step (a). The mixture may be watered by applying water to the depression.

In a second aspect of the disclosure, there is provided use of a composition comprising a compost composition, shredded almond hull and Trichoderma spp. spores in the preparation of Trichoderma spp. fungi.

In a third aspect of the disclosure, there is provided Trichoderma spp. fungi, whenever prepared by the method of the first aspect.

### Definitions

The following are some definitions that may be helpful in understanding the description of the present disclosure. These are intended as general definitions and should in no way limit the scope of the present disclosure to those terms alone, but are put forth for a better understanding of the following description.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The terms “a” and “an” are used herein to refer to one or to more than one (i.e. to at least one) of the grammatical object of the article. By way of example, “an element” means one element or more than one element.

In the context of this specification the term “about” is understood to refer to a range of numbers that a person of skill in the art would consider equivalent to the recited value in the context of achieving the same function or result.

Any numerical range recited herein is intended to include all sub-ranges of the same numerical precision subsumed within the recited range. For example, a range of 1.0 to 5.0 is intended to include all sub-ranges between (and including) the recited minimum value of 1.0 and the recited maximum value of 5.0, that is, having a minimum value equal to or greater than 1.0 and a maximum value equal to or less than 5.0, such as 2.1 to 4.5. Any maximum numerical limitation recited herein is intended to include all lower numerical limitations subsumed therein and any minimum numerical limitation recited herein is intended to include all higher numerical limitations subsumed therein.

### DETAILED DESCRIPTION

In one aspect of the disclosure there is provided a method for producing Trichoderma spp. fungi comprising
(i) combining shredded almond hull, a compost composition and Trichoderma spp. spores to form a mixture; and
(ii) incubating the mixture for a period of time sufficient to produce Trichoderma spp. fungi; and
(iii) collecting the Trichoderma spp. fungi.

The method provides an efficient means to produce beneficial Trichoderma spp. fungi utilising a readily available waste material, i.e. almond hull, as part of a growth medium. The method can be performed on a large scale and requires minimal capital cost.

The disclosure also relates to use of a composition comprising a compost composition, shredded almond hull and Trichoderma spp. spores in the preparation of Trichoderma spp. fungi.

The method may be used to prepare any species in the Trichoderma spp. genus, such as for example T. harzianum, T. viride, T. koningii or T. hamatum. In one embodiment the fungi is Trichoderma viride.

The Trichoderma spp. produced by the method may comprise both conidia and mycelia.

Without wishing to be bound by any particular theory, it is believed that the level of conidia produced is influenced by using shredded almond hull having a size between about 2 mm and about 5 mm. It was also found that the maximum conidia and mycelia count was observed where the almond hull to compost composition ratio was 10:1.

Step (i) involves preparation of a mixture comprising shredded almond hull, a compost composition and Trichoderma spp. spores. In order to facilitate mixing, the three components may be agitated. Agitation may comprise any suitable means that achieves mixing of the three components. In one embodiment, agitation comprises turning.

The compost composition acts as a source of nutrients (such as for example organic carbon, nitrogen, potassium and phosphorus) for growth of the fungi. The compost composition may serve to provide adequate nitrogen for fungal growth, and therefore balance the carbon:nitrogen ratio of the almond hull/compost composition combination.

Compost compositions useful in the method include those referred to colloquially and commercially as “mushroom compost”. Mushroom compost is typically produced by hot-composting organic matter, such as for example wheat straw, horse or poultry manure and gypsum. Other components such as corncobs, peat moss, stable bedding, cottonseed meal, canola meal, grape crushings from wineries, soybean meal, potash, urea, ammonium nitrate and lime, may also be present. In one embodiment, the compost composition is produced by composting a mixture comprising straw, horse or poultry manure and gypsum.

Compost compositions suitable for use in the method are commercially available. Suitable commercial compositions include All Purpose Mushroom Compost (Richgro® Green Products, Jandakot WA, Australia) and Premium Mushroom Compost (Searles® Garden Products, Kilcoy QLD, Australia). Alternatively, a suitable compost composition may be prepared as follows:
Step 1: Saturate 5 bales of straw with water and then drain for 24 hrs
Step 2: Begin assembling a compost heap by layering the straw with horse manure and gypsum. Layer one bale of straw, followed by 1 kg of gypsum, and 35 kg of manure.
Step 3: Repeat the layering process until the materials run out
Step 4: Leave the heap to break down naturally, and encourage this by mixing it every week with a shovel. It should be mixed in such a way that the outer materials are moved to the centre of the heap so that they can heat up.

Once the heap stops producing heat it is ready for use.

In one embodiment, the compost composition comprises one or more of the following:
a moisture content between about 50% (w/w) and 70% (w/w)
a pH between about 8.5 and about 10.5
an electrical conductivity between about 5 dS/m and about 20 dS/m.
a total carbon content between about 25% (w/w) and about 35% (w/w)
a total nitrogen content between about 1% (w/w) and about 5% (w/w)
a carbon:nitrogen ratio between about 5 and 15 (by weight)
organic matter in an amount between about 45% (w/w) and about 55% (w/w)
a nitrogen;sulfur ratio between about 1 and 2 (by weight)
a nitrogen:phosphorus ratio between about 2 and 4 (by weight)
a nitrogen;potassium ratio between about 0.5 and 2 (by weight)

In one embodiment the compost composition comprises the following parameters:

| | |
|---|---|
| Moisture content (%) | 62 |
| pH | 9.21 |
| Electrical conductivity | 9.77 |
| Total carbon | 31.4 |
| Total nitrogen | 2.78 |
| Carbon/nitrogen ratio | 11 |
| Estimated organic matter | 53.4 |
| Nitrogen/sulfur ratio | 1.6 |
| Nitrogen/phosphorus ratio | 2.9 |
| Nitrogen/potassium ratio | 1.1 |

The Trichoderma spp. spores may be provided in the form of the following commercially available products: Tricho-Shield™ (Nutri-Tech Solutions, Yandina, QLD, Australia), Trianum Shield (Tangsons Biotech Co., Ltd, Changsha, China), Tricho harzanium—Metcalf Gauntlet (Organic Crop Protectants, Clayton, VIC, Australia), Sabel-X® Hort (Sustainable Farming Solutions, Cottesloe, WA, Australia) and OGS Trichoderma fungi (available from organicgardeningsolutions.com.au).

Step (ii) involves incubating the mixture for a period of time sufficient to produce the Trichoderma spp fungi. The period of time may be between about 20 days and about 35 days, or between about 25 days and about 28 days. It has been found that a period of 25 to 28 days optimises mycelia and conidia production.

The method may further comprise watering the mixture. Watering may be performed prior to step (ii) and/or at one or more times during step (ii), depending on the moisture content of the mixture. It has been found that the moisture content of the mixture determines the growth rate and other physiological activities of Trichoderma spp. fungi produced by the method. It has been observed that fungal mycelia growth can occur across a wide range of moisture contents varying from 40% to 80%. Lower moisture levels were found to result in high conidia production. Where the mixture is saturated with moisture, the conditions become anaerobic which may reduce fungi proliferation. Where it is desired to balance conidia and mycelia production, the optimum moisture level was determined to be 55% to 65%.

The mixture may have a pH between about 6.6 and about 6.8. This pH range was found to optimise enzymatic activity and nutrient availability.

The temperature of the mixture also influences mycelia and conidia production. At higher temperatures (i.e. greater than 27° C.) increased mycelia production and reduced conidia production is observed. Conversely, at lower temperatures (i.e. below 25° C.) growth of both mycelia and conidia is slow. Where it is desired to maximise both conidia production and the ratio of mycelia:conidia, 25° C. to 27° C. was found to be the optimal temperature.

In some embodiments, the temperature and/or moisture content of the mixture is checked daily throughout step (ii) and adjusted as necessary in order to retain the desired moisture level and/or temperature. In some embodiments, water is added to the mixture on a weekly basis.

In one embodiment step (i) comprises:
(a) forming a layered structure comprising a first layer which is the shredded almond hull, a second layer which is the compost composition and a third layer which is the Trichoderma spp. spores, and agitating the layered structure to form the mixture.

In this embodiment the mixture is formed by first preparing a layered structure comprising each component and then agitating the layered structure to form the mixture. In one embodiment, the first layer represents a bottom layer, the second layer represents a middle layer and the third layer represents a top layer, such that the Trichoderma spp. spores are located on top of the compost composition which is located on top of the almond hull. The layered structure may be prepared by successively layering each component.

When forming the layered structure, the Trichoderma spp. spores may be applied manually, such as for example by hand.

In one embodiment, the mixture is in the form of one or more elongate piles. The elongate piles may resemble windrows used in composting methods.

In another embodiment the method further comprises forming a depression over a length of the elongate pile. When watering the mixture, water may be applied to the depression. In one embodiment the depression is formed along a top section of the elongate pile. In some embodiments the depression is formed about 5 to 10 days after completion of step (a).

Step (iii) may be performed after visually inspecting the development of the Trichoderma spp. fungi and the mycelia and conidia growth. Samples can then be sent for microbiological testing if desired.

The method may be performed on a large scale and is therefore capable of consuming large amounts of almond hull waste. The applicant has successfully performed the method using 300 metric tons of almond hull, as described in the examples section below.

An exemplary method in accordance with one embodiment of the disclosure is as follows:
Step 1: Almond hull was shredded into 2 mm to 5 mm pieces
Step 2: An elongate pile was prepared using 300 metric tons of the shredded almond hull
Step 3: 30 metric tons of a compost composition was added evenly along the top of the shredded almond hull using a front end loader
Step 4: 75 kg of Trichoderma spores (Tricho-Shield™ from Nutri-Tech Solutions, Yandina, QLD, Australia) was added manually by hand evenly over the compost composition to form a layered structure which resembled a windrow.
Step 5: The layered structure was then turned using a windrow turner and allowed to incubate
Step 6: After 1 week a depression about 75 cm to 100 cm deep was prepared along the entire length of the windrow
Step 7: The moisture level and temperature of the windrow were checked daily
Step 8: Once a week 12,000 to 15,000 litres of water were poured into the depression as required depending on the results of the moisture level checks
Step 9: After completion of the incubation period (which is typically 25 to 28 days) the fungi were visually inspected, collected and sent to a lab for a population test.

Trichoderma spp. fungi prepared in accordance with the disclosure may be used in the compost industry, as a growth additive in the nursery industry, or as a biological fungicide. Additional uses include, but are not limited to:
As a combination with compost to improve fungal population;
Application to soil in which vegetables, such as for example potato and carrots, are growing;
As a combination with biochar
Broadacre agriculture

In order to optimise its agronomic performance, the Trichoderma spp. fungi prepared in accordance with the disclosure should be stored in a cool, shady place out of direct sunlight. The moisture content should also be maintained at about 25% to 35% (w/w).

The method provides a sustainable means for producing Trichoderma spp. fungi using a waste material which may otherwise be discarded, thereby reducing the burden on waste disposal facilities.

### EXAMPLES

The present disclosure is further described below by reference to the following non-limiting example.

Production of Trichoderma spp.
Step 1: Almond hull was shredded into 2 mm to 5 mm pieces.
Step 2: A windrow was prepared with a front end loader using 300 metric tons of shredded almond hull having the following dimensions: width 3 m, length 92 m, height 0.9 m.
Step 3: 30 metric tons of mushroom compost (obtained from Peat Soil Ltd, Langhorne Creek, South Australia) was added evenly along the top of the almond hull windrow using a front end loader.
Step 4: 75 kg of Trichoderma spores (Tricho-Shield™ from Nutri-Tech Solutions, Yandina, QLD, Australia) was added manually by hand evenly over the windrow.
Step 5: The windrow was then turned using a windrow turner and allowed to incubate.
Step 6: After 1 week a depression about 75 cm to 100 cm deep was prepared along the entire length of the windrow.
Step 7: The moisture level and temperature of the windrow were checked daily after the depression was formed.
Step 8: Once a week 12,000 to 15,000 litres of water were poured into the depression as required depending on the results of the moisture level and temperature checks.
Step 9: After completion of the incubation period (which was about 25 days) the fungi were visually inspected, collected and sent to a lab for a population test.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of an two or more of said steps, features, compositions and compounds.

1. A method for producing Trichoderma spp. fungi comprising
(i) combining shredded almond hull, a compost composition and Trichoderma spp. spores to form a mixture; and
(ii) incubating the mixture for a period of time sufficient to produce Trichoderma spp. fungi; and
(iii) collecting the Trichoderma spp. fungi.

2. The method of claim 1, wherein the Trichoderma spp. fungi is T. harzianum, T. viride, T. koningii or T. hamatum.

3. The method of claim 1, wherein the Trichoderma spp. fungi produced comprise conidia and mycelia.

4. The method of claim 1, wherein the shredded almond hull, the compost composition and the Trichoderma spp. spores are present in the mixture in a ratio of about [0.5 to 1.5]:[0.05 to 0.15]:[0.000115 to 0.000375] by weight.

5. The method of claim 1, wherein the shredded almond hull has a size between about 1 mm and about 10 mm, or between about 2 mm and about 5 mm.

6. The method of claim 1, wherein the compost composition is a composition produced by composting a blend comprising straw, horse or poultry manure and gypsum.

7. The method of claim 1, wherein the compost composition comprises any one or more of the following:
a moisture content between about 50% (w/w) and 70% (w/w)
a pH between about 8.5 and about 10.5
an electrical conductivity between about 5 dS/m and about 20 dS/m.
a total carbon content between about 25% (w/w) and about 35% (w/w)
a total nitrogen content between about 1% (w/w) and about 5% (w/w)
a carbon:nitrogen ratio between about 5 and 15 (by weight)
organic matter in an amount between about 45% (w/w) and about 55% (w/w)
a nitrogen:sulfur ratio between about 1 and 2 (by weight)
a nitrogen:phosphorus ratio between about 2 and 4 (by weight)
a nitrogen:potassium ratio between about 0.5 and 2 (by weight)

8. The method of claim 1, wherein temperature and/or moisture content of the mixture is checked daily throughout step (ii)

9. The method of claim 1, further comprising watering the mixture.

10. The method of claim 9, wherein watering the mixture is performed one or more times during step (ii).

11. The method of claim 9, wherein watering the mixture is performed prior to commencing step (ii).

12. The method of claim 1, wherein the mixture has a pH between about 6.6 and about 6.8.

13. The method of claim 1, wherein the mixture has a temperature between about 25° C. and about 27° C.

14. The method of claim 1, wherein the mixture has a moisture content between about 40% and about 80% (w/w), or between about 55% and about 65% (w/w).

15. The method of claim 1, wherein the period of time is between about 20 days and about 35 days, or between about 25 days and about 28 days.

16. The method of claim 1, wherein step (i) comprises:
(a) forming a layered structure comprising a first layer which is the shredded almond hull, a second layer which is the compost composition and a third layer which is the Trichoderma spp. spores, and agitating the layered structure to form the mixture.

17. The method of claim 16, wherein the shredded almond hull forms a bottom layer, the compost composition forms a middle layer and the Trichoderma spp. spores form a top layer.

18. The method of claim 16, wherein the mixture is present as an elongate pile.

19. The method of claim 18, further comprising forming a depression over a length of the elongate pile.

20. The method of claim 19, wherein the depression is formed about 5 to 10 days after completion of step (a).
